(19) United States
(12) Patent Application Publication
(10) Pub. No.: US 2007/0060897 A1
(43) Pub. Date: Mar. 15, 2007

(54) Disposable safety syringe for dispensing anesthetic

(76) Inventor: Hsien Tsung Wang, Taipei (TW)

Correspondence Address: BRUCE H. TROXELL, SUITE 1404, 5205 LEESBURG PIKE, FALLS CHURCH, VA, 22041, US

(21) Appl. No.: 11/217,373

(22) Filed: Sep. 2, 2005

(51) Int. Cl. A61M 5/00

(52) U.S. Cl. 604/232

(57) ABSTRACT

A safety syringe for dispensing anesthetic is provided. After a dispensing operation pulling a plunger rearward will retract a needle assembly into a syringe barrel until a projection on a surface of the plunger moves from a forward end of an L-shaped slot to a rear square opening by passing under a stop for locking the projection in the square opening. The syringe is thus disposable and thereby prevents the used syringe from being used again by sterilizing the syringe.

### BACKGROUND OF THE INVENTION

1. Field of Invention

The present invention relates to syringes and more particularly to a syringe for dispensing anesthetic with safety arrangement for concealing and locking a needle after use such that the syringe is disposable and thereby prevents the used syringe from being used again by sterilizing the syringe.

2. Description of Related Art

Conventionally, a syringe for dispensing anesthetic is made of stainless steel and which is relatively high in cost. Thus, it is typical of sterilizing the used syringe rather than discarding it. However, in often times microorganisms may be not completely killed in the sterilization. As to the sterilization, it involves steps of removing a needle from a syringe and sterilizing the syringe thereafter. However, it is highly possible that the needle may accidentally prick fingers of a medical worker and thereby contaminate the medical worker with microorganisms. However, so far as the present inventor is aware, there is no such syringe available on the market that completely and satisfactorily solves the safety problem. Thus, the need for improvement still exists in order to overcome the inadequacy of the prior art.

### SUMMARY OF THE INVENTION

It is therefore an object of the present invention to provide a safety syringe for dispensing anesthetic in which after a dispensing operation pulling a plunger rearward will retract a needle assembly into a syringe barrel until the projection moves from a forward end of an L-shaped slot to a rear square opening by passing under a stop for locking the projection in the square opening. The syringe is thus disposable and thereby prevents the used syringe from being used again by sterilizing the syringe.

To achieve the above and other objects, the present invention provides a retractable syringe for dispensing anesthetic comprising a fluid cartridge including a rear sliding member; an elongated arm including a rear ring for enabling a finger to insert into, and a forward disk having a diameter slightly smaller than that of the sliding member; a hollow, cylindrical plunger for receiving the fluid cartridge and including a projection on its outer surface proximate a forward end, a plurality of outer threads at a rear end, and a forward threaded nose; a wing nut including a forward hollow enlargement having a plurality of inner threads threadedly secured to the outer threads of the plunger for fastening the wing nut and the plunger together, and an axial rear channel such that the arm is adapted to insert through the rear channel and the enlargement into the plunger to engage the sliding member; a needle assembly including a housing having inner threads threadedly secured to the nose, a needle extended forward from the housing, and a slender metal tube extended rearward from a rear end of the needle into the fluid cartridge for forming a fluid path from the fluid cartridge to the needle via the metal tube; and an elongated syringe barrel including a forward hollow extension, a rear square opening, and a lengthwise slot on its surface, the slot including a stop for separating the square opening from the slot; whereby in an anesthetic dispensing operation pushing the arm forward will project the needle from the syringe barrel and move the projection to a forward end of the slot, and after the dispensing operation pulling the plunger rearward will retract the needle assembly into the syringe barrel until the projection moves from the forward end of the slot to the square locking opening by passing under the stop for locking the projection in the square opening.

In one aspect of the present invention the syringe barrel further a rear elongate groove on its inner surface, the groove being adapted to guide the projection into the slot in assembly.

In another aspect of the present invention the transverse portion of the slot is formed as an opening adapted to receive the projection in a fluid cartridge replacement operation.

The above and other objects, features and advantages of the present invention will become apparent from the following detailed description taken with the accompanying drawings.

### BRIEF DESCRIPTION OF THE DRAWINGS

FIG. 1 is a perspective view of a first preferred embodiment of syringe for dispensing anesthetic according to the invention;

FIG. 1A is a detailed view of the area in circle A in FIG. 1;

FIG. 2 is an exploded perspective view of the syringe of FIG. 1;

FIG. 3 is a longitudinal view of the syringe in FIG. 1 where the needle is projected in a fluid dispensing operation;

FIG. 4 is a longitudinal sectional view of the syringe in FIG. 3;

FIG. 4A is a detailed view of the area in an upper circle in FIG. 4;

FIG. 4B is a detailed view of the area in a lower circle in FIG. 4;

FIG. 5 is a longitudinal view of the syringe in FIG. 1 where the needle is concealed to be ready to be discarded after use;

FIGS. 6A, 6B, 6C, and 6D are enlarged sectional views taken along line 6-6 of FIG. 3 showing four different configurations of projection and stop;

FIG. 7 is a perspective view of a second preferred embodiment of syringe for dispensing anesthetic according to the invention;

FIG. 8 is an exploded perspective view of the syringe of FIG. 7;

FIG. 9 is a longitudinal view of the syringe in FIG. 7 where the needle is projected in a fluid dispensing operation;

FIG. 9A is a detailed view of the area in a circle in FIG. 9;

FIG. 10 is a longitudinal sectional view of the syringe in FIG. 9; and

FIG. 11 is a longitudinal view of the syringe in FIG. 7 where the needle is concealed to be ready to be discarded after use.

### DETAILED DESCRIPTION OF THE INVENTION

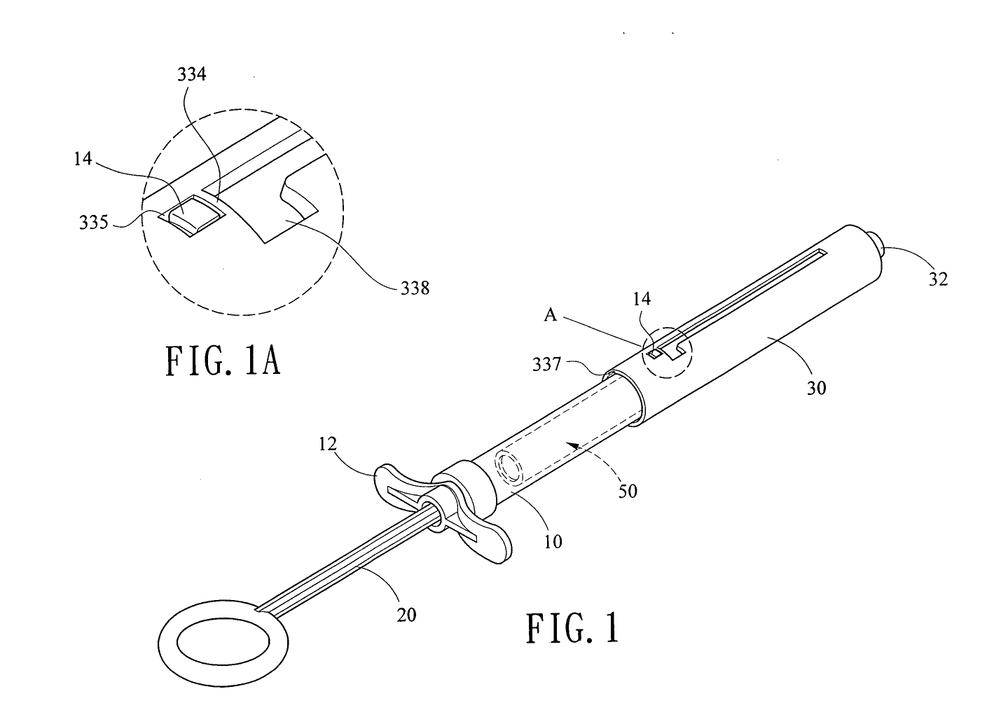

Referring to FIGS. 1 to 5 and 6A to 6D, a disposable safety syringe for dispensing anesthetic in accordance with a first preferred embodiment of the invention comprises a plunger 10, an arm 20, a syringe barrel 30, a hypodermic needle assembly 40, and a fluid cartridge 50. Each component is discussed in detailed below.

The fluid cartridge 50 is a cylindrical container made of glass and is commercially available. The fluid cartridge 50 comprises a forward extension 51 formed of flexible material, and a cylindrical sliding member 52 provided in a rear end in a fully charged state of the fluid cartridge 50 and adapted to slide toward the extension 51 for extracting fluid from a space defined by the extension 51 and the sliding member 52 in a fluid dispensing operation.

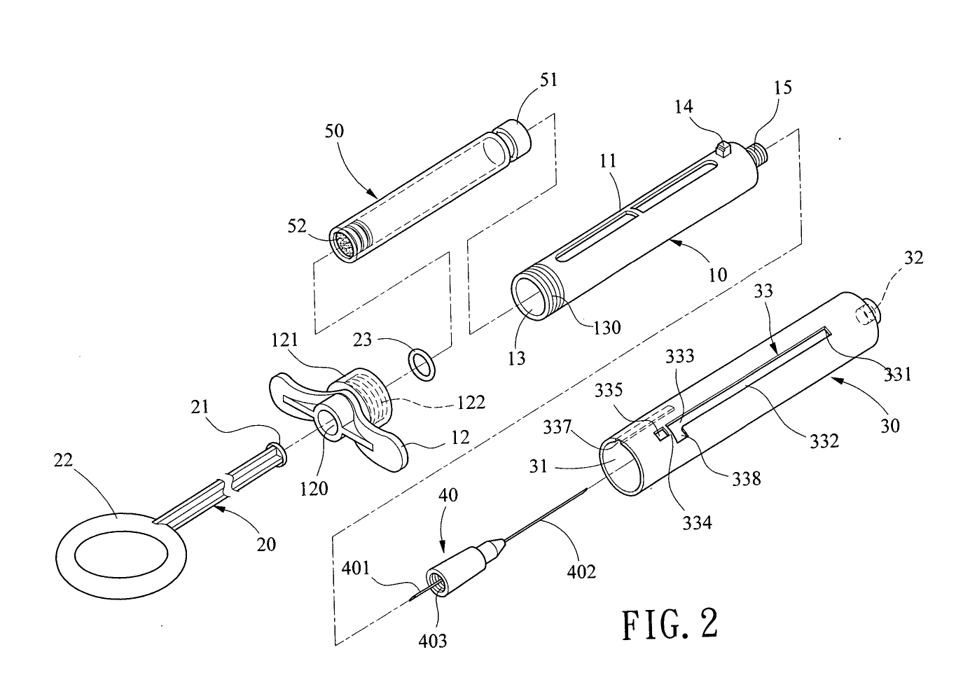

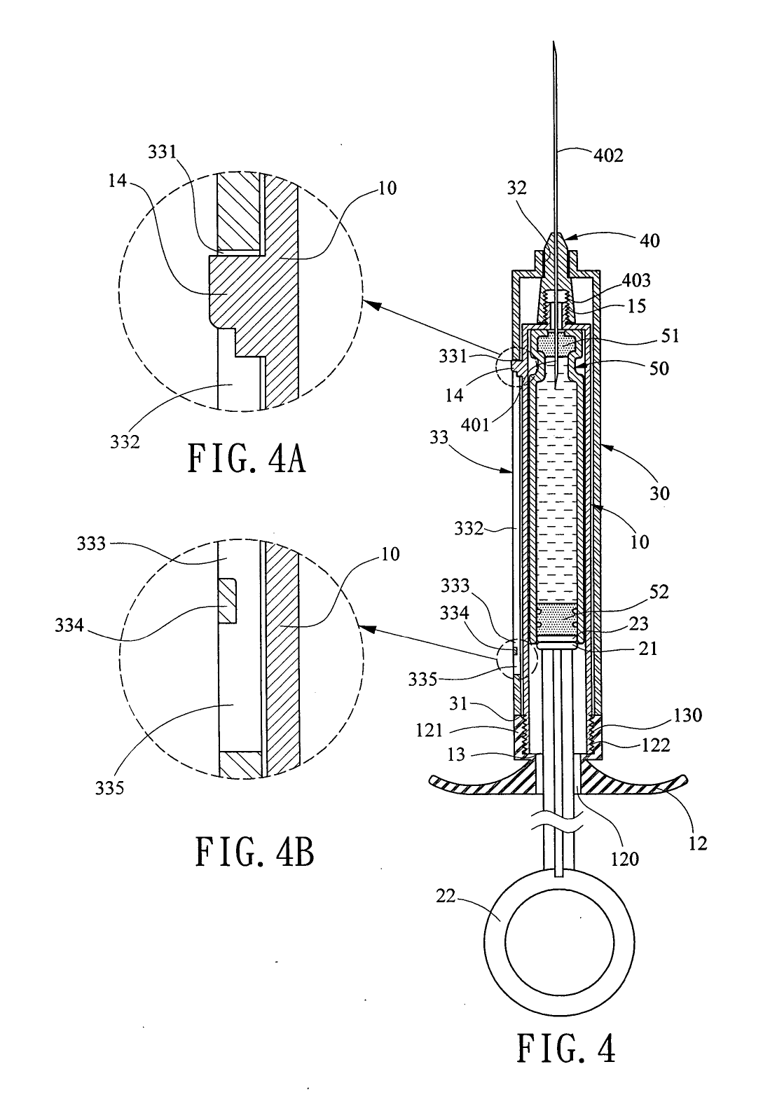

The hollow, cylindrical plunger 10 is formed of a plastic material and comprises an internal space 11 for receiving the fluid cartridge 50 therein, a rear end 13 having outer threads 130, a forward nose 15 having a diameter smaller than that of the main body of the plunger 10 and outer threads, and a projection 14 on its outer surface proximate the nose 15 in which as shown in FIGS. 2 and 4A, the projection 14 has a transverse section of square having a higher forward side (i.e., a top surface of the projection 14 has a higher portion proximate the nose 15 and a lower portion distal the nose 15).

The plunger 10 further comprises a separate wing nut 12 including an axial bore 120 and a forward hollow, cylindrical enlargement 121 having inner threads 122 threadedly secured to the outer threads 130 for fastening the wing nut 12 and the plunger 10 together.

The arm 20 is elongate and comprises a rear ring 22 as a handle for enabling a finger to insert into, a forward disk 21 having a diameter slightly smaller than that of the sliding member 52, and a separate rubber ring 23. The arm 20 is adapted to insert through the bore 120, the enlargement 121, the rubber ring 23, and a rear portion of the space 11 to urge against the sliding member 52 in which the rubber ring 23 is disposed on the shank of the arm 20 and engages with the disk 21 for preventing the arm 20 from disengaging with the wing nut 12 in pushing or pulling operation of the arm 20 by holding the ring 22 while dispensing anesthetic.

The needle assembly 40 comprises a rear conic housing having inner threads 403 threadedly secured to the threaded nose 15 for fastening the needle assembly 40 and the plunger 10 together, a needle 402 extended forward from the housing, and a slender metal tube 401 extended rearward from a rear end of the needle 402 in the housing through the extension 51 into the fluid cartridge 50 such that fluid communication from the fluid cartridge 50 to the needle 402 via the metal tube 401 can be effected.

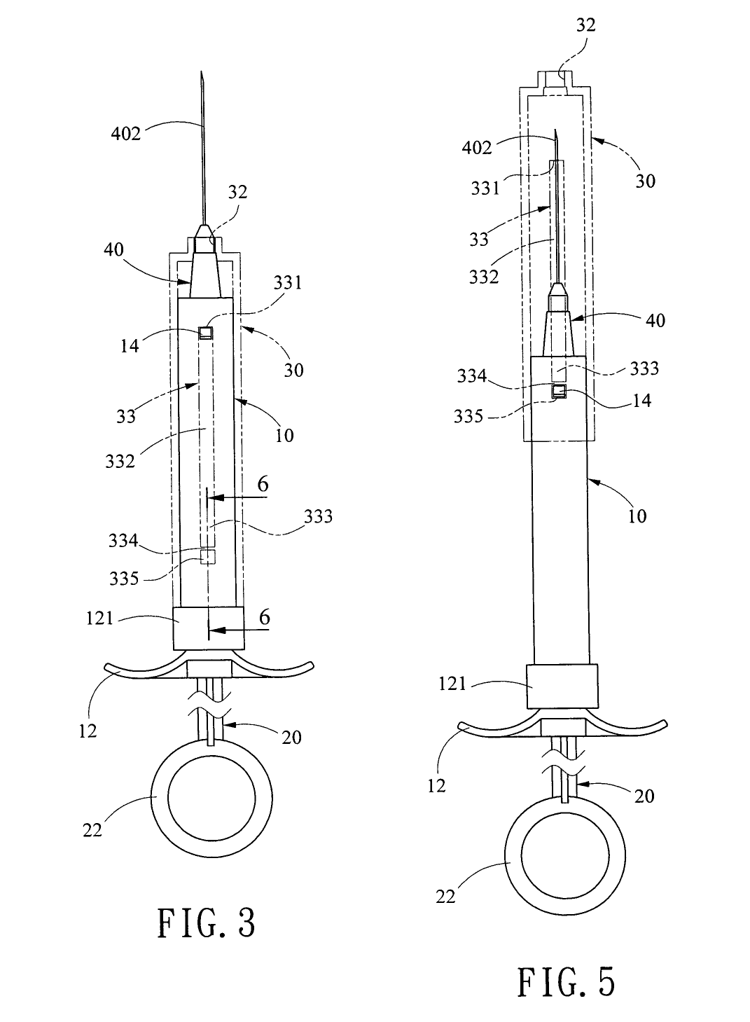

The syringe barrel 30 is an elongate, cylindrical member made of a plastic material and comprises an axial bore 31 having a diameter slightly larger than an outer diameter of the plunger 10 such that the assembled needle assembly 40 and the plunger 10 can be disposed in the bore 31 (see FIGS. 3 and 4). The syringe barrel 30 further comprises a forward hollow extension 32, and a lengthwise slot 33 on its surface. The slot 33 comprises a forward end 331, a rear end 333, a straight portion 332 between the forward and the rear ends 331 and 333, a rear square locking opening 335 at the rear of the rear end 333, a protruded opening 338 at one side of the rear end 333, the protruded opening 338 being adapted to receive the projection 14 in a fluid cartridge 50 replacement operation, and a stop 334 for separating the square locking opening 335 from the protruded opening 338. The syringe barrel 30 further comprises a rear elongate groove 337 on its inner surface extending from a rear end of the barrel to an inner portion of the barrel. The groove 337 is adapted to guide the projection 14 into the slot 33 in assembly.

The projection 14 is adapted to be movably disposed in the slot 33 and to be stopped in the rear end 333 in an unused state of the syringe and to be locked in the protruded opening 338 and the needle 402 is thus concealed in the syringe barrel 30 for preventing a medical worker from being contaminated with microorganisms due to accidental pricking. In a fluid dispensing operation, a medical worker may use one hand to hold the syringe barrel 30 and use the other hand to push the arm 20 forward for projecting the needle 402 from the forward extension 32 with the projection 14 moved forward from the rear end 333 along the straight portion 332 until being stopped at the forward end 331. At the same time, the needle 402 inserts into the body of a patient. After dispensing fluid (e.g., anesthetic), a medical worker can pull the plunger 10 rearward (i.e., the syringe barrel 30 moves forward) to retract the needle assembly 40 into the syringe barrel 30 with the projection 14 moved rearward from the forward end 331 along the straight portion 332 until being stopped at the rear end 333. At this time, the medical worker may further pull the plunger 10 rearward to cause the projection 14 to pass under the stop 334 prior to entering the square locking opening 335. The needle assembly 40 is thus locked in the syringe barrel 30 as the stop 334 is projected from the square locking opening 335. The whole syringe is thus no longer useful and thereby prevents the used syringe from being re-used again by sterilizing the syringe.

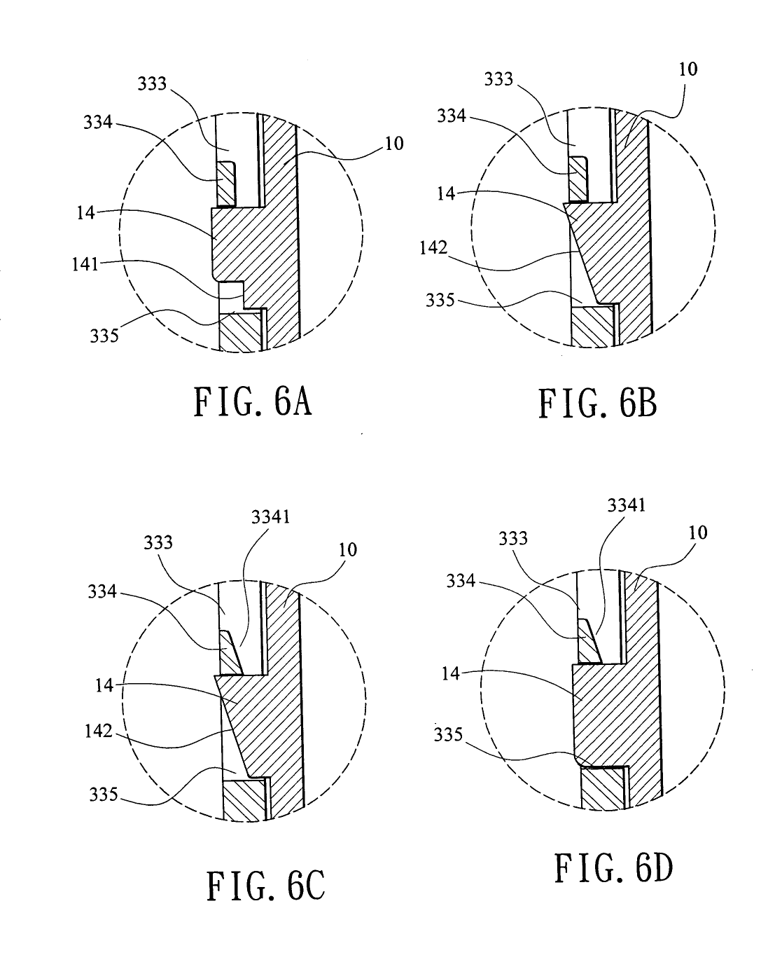

Referring to FIGS. 6A, 6B, 6C, and 6D specifically, four different configurations of the projection 14 and the stop 334 are shown. In FIG. 6A, a top surface of the projection 14 has a higher portion proximate the stop 334 and a lower portion 141 distal the stop 334 in its locked position in the square opening 335. In FIG. 6B, a top surface 142 of the projection 14 is slanted downward rearward. In FIG. 6C, the stop 334 has a bottom surface 3341 slanted downward rearward (i.e., the stop 334 has a trapezoidal section with a forward edge being smaller than a rear edge). In FIG. 6D, the stop 334 has the same shape as that shown in FIG. 6C and the projection 14 has a flat top surface. All of the above configurations of the projection 14 and the stop 334 aim at facilitating the projection 14 to pass under the stop 334 prior to entering the square locking opening 335 for locking.

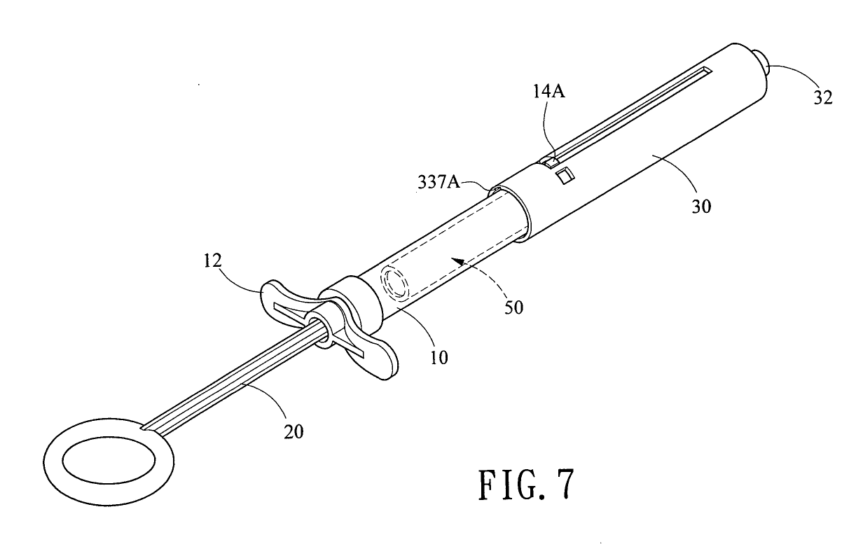

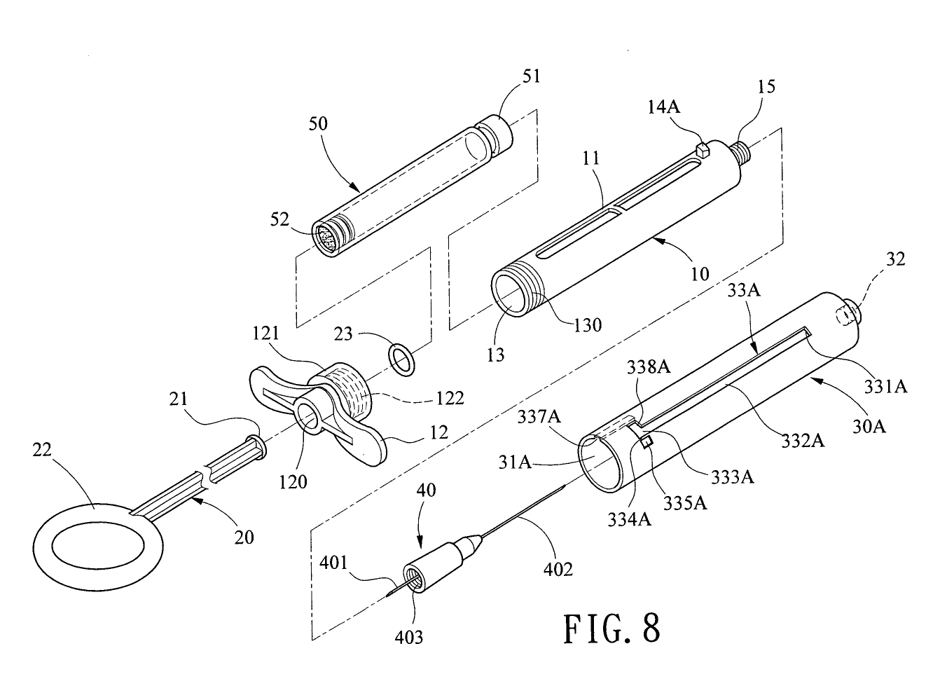

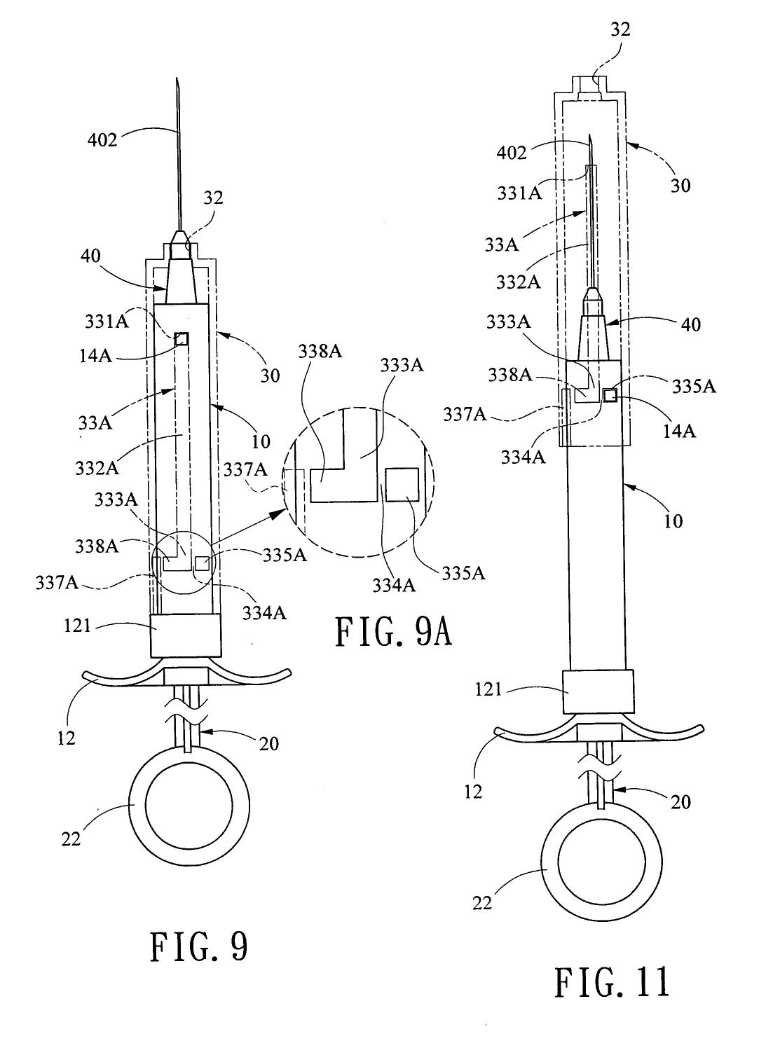

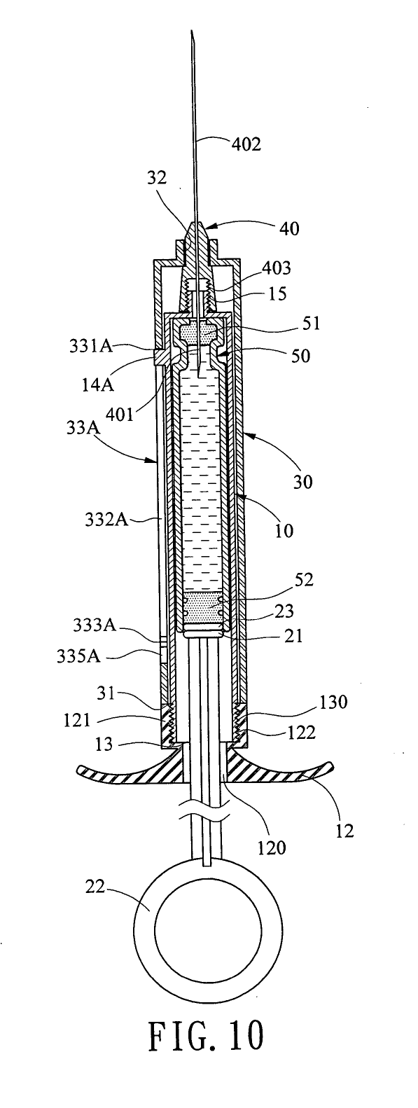

Referring to FIGS. 7 to 11, a disposable safety syringe for dispensing anesthetic in accordance with a second preferred embodiment of the invention is shown. The second preferred embodiment substantially has same structure as the first preferred embodiment. The differences between the first and the second preferred embodiments, i.e., the characteristics of the second preferred embodiment are detailed below.

The syringe barrel 30A comprises an axial bore 31A having a diameter slightly larger than an outer diameter of the plunger 10 such that the assembled needle assembly 40 and the plunger 10 can be disposed in the bore 31A. The syringe barrel 30A further comprises a forward hollow extension 32, and a lengthwise revised L-shaped slot 33A on its surface. The slot 33A comprises a forward end 331A, a rear end 333A, a straight portion 332A between the forward and the rear ends 331A and 333A, a rear square locking opening 335A at one side of the rear end 333A, a protruded opening 338A at the other side of the rear end 333A opposite the square locking opening 335A, the protruded opening 338A being adapted to receive the projection 14A in a fluid cartridge 50 replacement operation, and a stop 334A for separating the square locking opening 335A from the protruded opening 338A. The syringe barrel 30A further comprises a rear elongate groove 337A on its inner surface extending from a rear end of the barrel to an inner portion of the barrel. The groove 337A is adapted to guide the projection 14A into the slot 33A in assembly.

The projection 14A is adapted to be movably disposed in the slot 33A and to be stopped in the rear end 333A in an unused state of the syringe and the needle 402 is thus concealed in the syringe barrel 30A for preventing a medical worker from being contaminated with microorganisms due to accidental pricking. In a fluid dispensing operation, a medical worker may use one hand to hold the syringe barrel 30A and use the other hand to push the arm 20 forward for projecting the needle 402 from the forward extension 32 with the projection 14A moved forward from the rear end 333A along the straight portion 332A until being stopped at the forward end 331A. At the same time, the needle 402 inserts into the body of a patient. After dispensing fluid (e.g., anesthetic), the medical worker can pull the plunger 10 rearward (i.e., the syringe barrel 30A moves forward) to retract the needle assembly 40 into the syringe barrel 30A with the projection 14A moved rearward from the forward end 331A along the straight portion 332A until being stopped at the rear end 333A. At this time, the medical worker may further turn the plunger 10 rightward to cause the projection 14A to pass under the stop 334A prior to entering the square locking opening 335A. The needle assembly 40 is thus locked in the syringe barrel 30A as the stop 334A is projected from the square locking opening 335A. The whole syringe is thus no longer useful and thereby prevents the used syringe from being re-used again by sterilizing the syringe.

While the invention herein disclosed has been described by means of specific embodiments, numerous modifications and variations could be made thereto by those skilled in the art without departing from the scope and spirit of the invention set forth in the claims.

What is claimed is:

1. A retractable syringe for dispensing anesthetic comprising:
a fluid cartridge including a rear sliding member;
an elongated arm including a rear ring for enabling a finger to insert into, and a forward disk having a diameter slightly smaller than that of the sliding member;
a hollow, cylindrical plunger for receiving the fluid cartridge and including a projection on its outer surface proximate a forward end, a plurality of outer threads at a rear end thereof, and a forward threaded nose;
a wing nut including a forward hollow enlargement having a plurality of inner threads threadedly secured to the outer threads of the plunger for fastening the wing nut and the plunger together, and an axial rear channel such that the arm is adapted to insert through the rear channel and the enlargement into the plunger to engage the sliding member of the cartridge;
a needle assembly including a housing having inner threads threadedly secured to the nose of the plunger, a needle extended forward from the housing, and a slender metal tube extended rearward from a rear end of the needle into the fluid cartridge for forming a fluid path from the fluid cartridge to the needle via the metal tube; and
an elongated syringe barrel including a forward opening, a rear locking opening, and a lengthwise slot on its surface, the slot including a stop for separating the locking opening from the slot;
whereby in an anesthetic dispensing operation pushing the arm forward will project the needle from the syringe barrel and move the projection to a forward end of the slot, and after the dispensing operation pulling the plunger rearward will retract the needle assembly into the syringe barrel until the projection moves from the forward end of the slot to the locking opening by passing under the stop for locking the projection in the locking opening.

2. The retractable syringe of claim 1, wherein the lengthwise slot of the syringe barrel further comprises a forward end, a rear end, a straight portion between the forward and the rear ends, a rear locking opening at the rearmost of the rear end, a protruded opening at one side of the rear end.

3. The retractable syringe of claim 1, wherein the projection has a transverse section of square, and a top surface of the projection has a higher portion proximate the nose and a lower portion distal the nose.

4. The retractable syringe of claim 1, wherein the stop has a trapezoidal longitudinal section with its forward edge being smaller than its rear edge.

5. The retractable syringe of claim 1, wherein a top surface of the projection is slanted downward rearward.

6. The retractable syringe of claim 1, wherein the plunger further comprises a separate rubber ring disposed in close proximity to a rear of the disk.

7. The retractable syringe of claim 1, wherein the syringe barrel further comprises a rear elongate groove on its inner surface extending from a rear end of the barrel to an inner portion of the barrel, the groove being adapted to guide the projection into the slot in assembly.

8. The retractable syringe of claim 1, wherein the slot further comprises a protruded opening at its rear end, the protruded opening being adapted to receive the projection in a fluid cartridge replacement operation.

9. The retractable syringe of claim 1, wherein the rear locking opening of the syringe barrel is of square.

10. A retractable syringe for dispensing anesthetic comprising:
a fluid cartridge including a rear sliding member;
an elongated arm including a rear ring for enabling a finger to insert into, and a forward disk having a diameter slightly smaller than that of the sliding member;
a hollow, cylindrical plunger for receiving the fluid cartridge and including a projection on its outer surface proximate a forward end, a plurality of outer threads at a rear end, and a forward threaded nose;
a wing nut including a forward hollow enlargement having a plurality of inner threads threadedly secured to the outer threads of the plunger for fastening the wing nut and the plunger together, and an axial rear channel such that the arm is adapted to insert through the rear channel and the enlargement into the plunger to engage the sliding member;
a needle assembly including a housing having inner threads threadedly secured to the nose of the plunger, a needle extended forward from the housing, and a slender metal tube extended rearward from a rear end of the needle into the fluid cartridge for forming a fluid path from the fluid cartridge to the needle via the metal tube; and
an elongated syringe barrel including a forward hollow extension, a rear locking opening, and a lengthwise slot on its surface, a transverse portion of the slot being opposite the locking opening, an the slot including a stop for separating the locking opening from the slot;
whereby in an anesthetic dispensing operation pushing the arm forward will project the needle from the syringe barrel and move the projection to a forward end of the slot, and after the dispensing operation pulling the plunger rearward will retract the needle assembly into the syringe barrel until the projection moves from the forward end of the slot to the locking opening by passing under the stop for locking the projection in the locking opening.

11. The retractable syringe of claim 10, wherein the lengthwise slot of the syringe barrel further comprises a forward end, a rear end, a straight portion between the forward and the rear ends, a rear locking opening at one side of the rear end, a protruded opening at the other side of the rear end opposite the locking opening.

12. The retractable syringe of claim 10, wherein the projection has a transverse section of square, and a top surface of the projection has a higher portion proximate the nose and a lower portion distal the nose.

13. The retractable syringe of claim 10, wherein each of the needle assembly, the arm, and the syringe barrel is formed of plastics.

14. The retractable syringe of claim 10, wherein the stop has a trapezoidal longitudinal section with its forward edge being smaller than its rear edge.

15. The retractable syringe of claim 10, wherein a top surface of the projection is slanted downward rearward.

16. The retractable syringe of claim 10, wherein the plunger further comprises a separate rubber ring disposed in close proximity to a rear of the disk.

17. The retractable syringe of claim 10, wherein the syringe barrel further comprises a rear elongate groove on its inner surface extending from a rear end of the barrel to an inner portion of the barrel, the groove being adapted to guide the projection into the slot in assembly.

18. The retractable syringe of claim 10, wherein the transverse portion of the slot is formed as an opening adapted to receive the projection in a fluid cartridge replacement operation.

19. The retractable syringe of claim 10, wherein the rear locking opening of the syringe barrel is of square.
